(19) United States
(12) Patent Application Publication
(10) Pub. No.: US 2019/0008796 A1
(43) Pub. Date: Jan. 10, 2019

(54) FORMULA AND PRODUCT TO COMBAT CANNABIS INDUCED PARANOIA AND/OR ANXIETY

(71) Applicant: John C. Darwin, Denver, CO (US)

(72) Inventor: John C. Darwin, Denver, CO (US)

(21) Appl. No.: 16/031,038

(22) Filed: Jul. 10, 2018

Related U.S. Application Data
(60) Provisional application No. 62/530,603, filed on Jul. 10, 2017.

(51) Int. Cl. A61K 31/015; A61K 36/185

(52) U.S. Cl. CPC A61K 31/015; A61K 36/185

(57) ABSTRACT

A system for combatting cannabis induced paranoia and anxiety, the system includes a product to be ingested by a user; and a terpene mixture to be incorporated into the product, the terpene mixture having an amount of alpha pinene; an amount beta caryophyllene; and an amount of limonene; the terpene mixture is integrally mixed into the product at a predetermined percentage; and the product is to be ingested to combat cannabis induced paranoia and/or anxiety.

### BACKGROUND

### 1. Field of the Invention

The present invention relates generally to cannabis use for recreation and medical purposes, and more specifically, to a formula and method to make a product for combating cannabis induced paranoia and/or anxiety.

### 2. Description of Related Art

Ingestion of cannabis, either through edible cannabis products, smoking, vaping, or other means, is common and provides beneficial medicinal and recreational effects. One of the problems commonly associated with the use of cannabis products is cannabis induced paranoia. It is understood that such paranoia is caused by cannabinoids, such as THC, binding to receptors throughout the brain. Many of these receptors are involved in emotional processing, governing responses such as fear, stress, anxiety, and paranoia. It is understood that said cannabinoids can overexcite the neural pathways and lead to anxiety and paranoia, especially in individuals who are new or unaccustomed to cannabis.

Accordingly, it is desirous to create a product that is natural and/or organic, that provides relief for the symptoms of paranoia and/or anxiety and is also safe to use with cannabis products.

### DESCRIPTION OF THE DRAWINGS

The novel features believed characteristic of the embodiments of the present application are set forth in the appended claims. However, the embodiments themselves, as well as a preferred mode of use, and further objectives and advantages thereof, will best be understood by reference to the following detailed description when read in conjunction with the accompanying drawings, wherein:

FIG. 1 is a simplified schematic of a formula associated with ingredients desirable for a product in accordance with a preferred embodiment of the present application;

FIG. 2 is a flowchart of the method associated with creating the product of FIG. 1;

FIG. 3 is a simplified schematic of an edible product in accordance with one embodiment of the product of FIG. 1;

FIG. 4 is a simplified schematic of a distillate in accordance with an alternative embodiment of the product of FIG. 1;

FIG. 5 is a simplified schematic of a capsule in accordance with an alternative embodiment of the product of FIG. 1; and

FIG. 6 is a simplified schematic of a powder in accordance with an alternative embodiment of the product of FIG. 1.

While the system and method of use of the present application is susceptible to various modifications and alternative forms, specific embodiments thereof have been shown by way of example in the drawings and are herein described in detail. It should be understood, however, that the description herein of specific embodiments is not intended to limit the invention to the particular embodiment disclosed, but on the contrary, the intention is to cover all modifications, equivalents, and alternatives falling within the spirit and scope of the present application as defined by the appended claims.

### DETAILED DESCRIPTION OF THE PREFERRED EMBODIMENT

Illustrative embodiments of the system and method of use of the present application are provided below. It will of course be appreciated that in the development of any actual embodiment, numerous implementation-specific decisions will be made to achieve the developer's specific goals, such as compliance with system-related and business-related constraints, which will vary from one implementation to another. Moreover, it will be appreciated that such a development effort might be complex and time-consuming, but would nevertheless be a routine undertaking for those of ordinary skill in the art having the benefit of this disclosure.

The system and method of the present invention provides a means to naturally combat the effects of cannabis induced paranoia and/or anxiety. These and other unique features of the system and method of use are discussed below and illustrated in the accompanying drawings.

The system and method of use will be understood, both as to its structure and operation, from the accompanying drawings, taken in conjunction with the accompanying description. Several embodiments of the system are presented herein. It should be understood that various components, parts, and features of the different embodiments may be combined together and/or interchanged with one another, all of which are within the scope of the present application, even though not all variations and particular embodiments are shown in the drawings. It should also be understood that the mixing and matching of features, elements, and/or functions between various embodiments is expressly contemplated herein so that one of ordinary skill in the art would appreciate from this disclosure that the features, elements, and/or functions of one embodiment may be incorporated into another embodiment as appropriate, unless described otherwise.

The preferred embodiment herein described is not intended to be exhaustive or to limit the invention to the precise form disclosed. It is chosen and described to explain the principles of the invention and its application and practical use to enable others skilled in the art to follow its teachings.

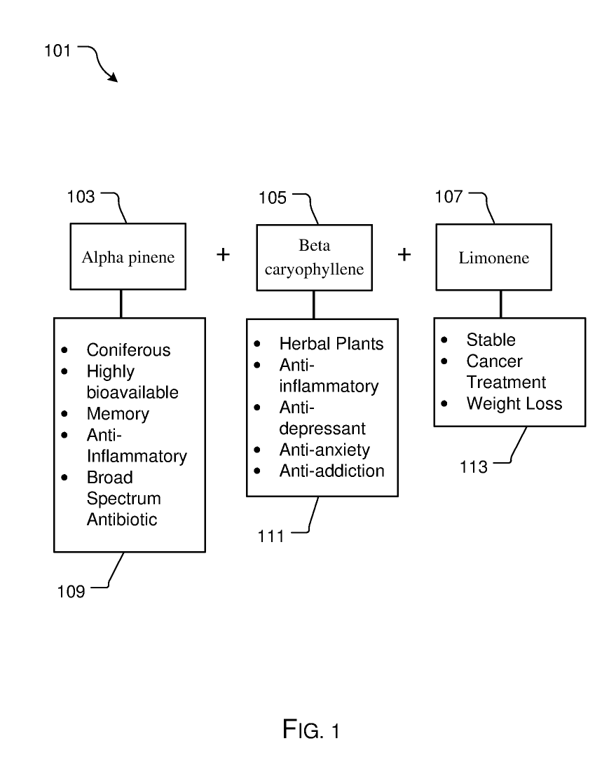

Referring now to the drawings wherein like reference characters identify corresponding or similar elements throughout the several views, FIG. 1 depicts a simplified paranoia and/or anxiety combatting formula 101 in accordance with a preferred embodiment of the present application. It will be appreciated that formula 101 overcomes one or more of the above-listed problems commonly associated with conventional cannabis ingestion systems.

In the contemplated embodiment, formula 101 includes a combination of three terpenes, namely alpha pinene 103, beta caryophyllene 105, and limonene 107. It should be understood that terpenes are a diverse class of organic compounds, produced primarily by plants, but can be synthetically created as well. Terpenes typically produce an odor and can have various neurological and/or biological effect on a user when ingested. Many terpenes, such as the three discussed herein, are considered natural treatments for many biological conditions. In the present invention, it should be understood that the combination of the aforementioned terpenes (alpha pinene, beta caryophyllene, and limonene) have been found to provide the beneficial effect of reducing paranoia and/or anxiety in individuals. It should be understood that this feature is specifically desirable during or after the ingestion of cannabis or cannabis products for recreational or medical purposes. It should be appreciated that “ingestion” includes vaporizing, eating, smoking, topical application, and any other means.

As shown in FIG. 1, alpha pinene 103, includes the following characteristics 109. Alpha Pinene is an organic compound having two isomers of pinene and is most notably found in oils from coniferous trees. Literature suggests that this terpene is highly bioavailable within human body at low exposer levels, and has been shown to aid in memory, work as an anti-inflammatory, and as a broad spectrum antibiotic.

Beta caryophyllene 105, includes the following characteristics 111. Beta caryophyllene is particularly associated with herbal plants such as rosemary, hops, cloves, cannabis, and black pepper and has been found to aid in treatment of pain, inflammation, depression, anxiety, addiction, and epilepsy.

Limonene 107, includes the following characteristics 113. Limonene is primarily derived from citrus fruit and is considered a stable terpene and has shown to aid in cancer treatment, weight loss, and other conditions.

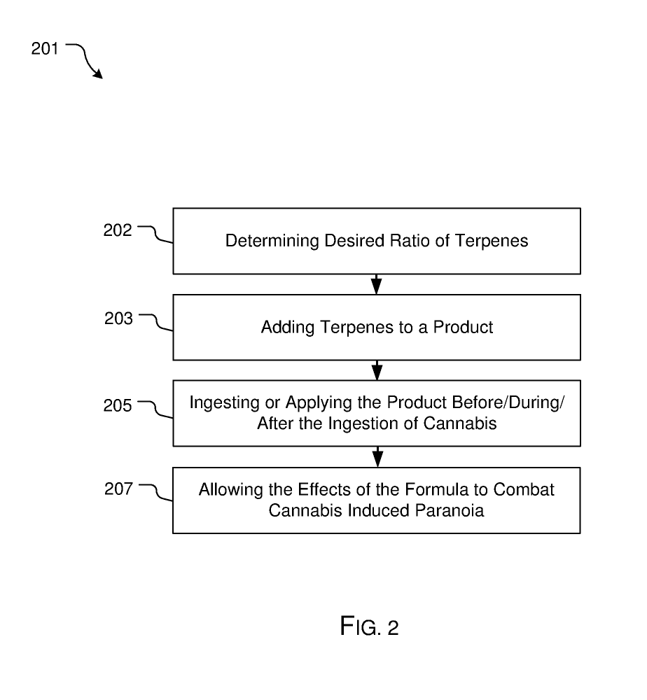

In FIG. 2, a flowchart 201 depicts the method associated with formula 101. During use, a desired ratio of the aforementioned terpenes is selected, as shown with box 202. It should be appreciated that although a relatively even ratio is desirable, various embodiments can include minor alterations. Alpha pinene, beta caryophyllene, and limonene extracts are added to a product at a predetermined ratio, as shown with box 203. The product is consumed before, during, or after the ingestion of a cannabis product, as shown with box 205. As cannabis induced paranoia or anxiety begins to set into the user, the terpenes ingested begin to calm and settle the user through their unique chemical compositions, as shown with box 207.

It should be appreciated that in one embodiment, the above terpenes are added to the product in an equal ratio, however, it is contemplated that the ratios can be modified as desired for taste and aroma of the product. It should be appreciated that due to the fact that the terpenes are primarily natural, their exact proportions can be altered with limited effect on a user.

In FIG. 3, a simplified schematic 301 shows one embodiment of the product associated with method 201, wherein the product is an edible cannabis product 302, thereby creating a product wherein the terpenes 303, 305, 307 associated with the above formula are ingested during the ingestion of cannabis. It should be appreciated that an edible cannabis product can be a candy, a drink, a bakery product, or any other cannabis edible.

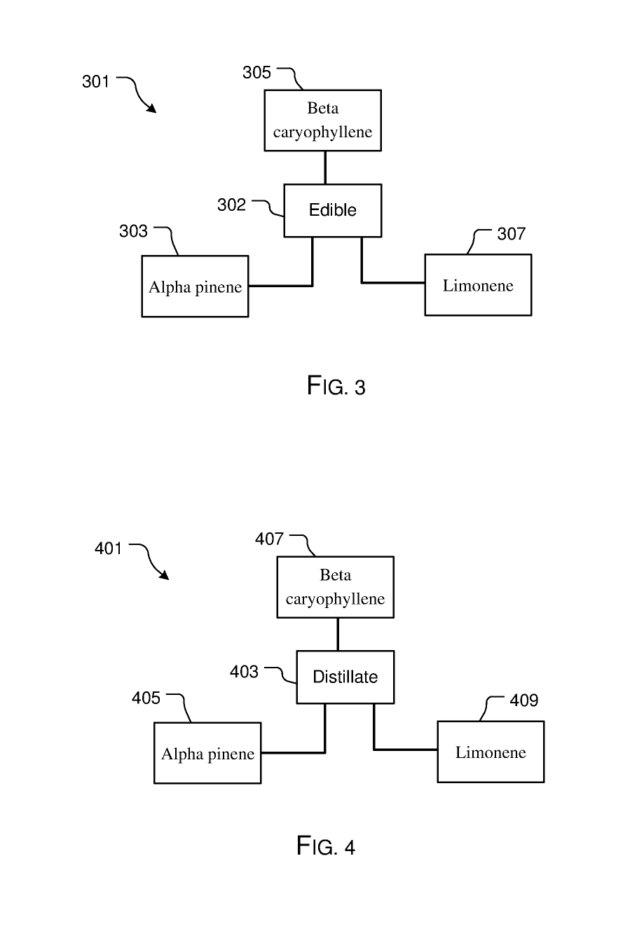

In FIG. 4, another simplified schematic 401 shows another embodiment of the product associated with method 201, wherein the product is a cannabis distillate 403, and wherein the terpenes 405, 407, 409 are added through dropping the terpenes directly into the distillate oil. It should be appreciated that although various amounts of each terpene can be added, in the preferred embodiment, the amount is 1-2 drops per 1 gram of distillate oil.

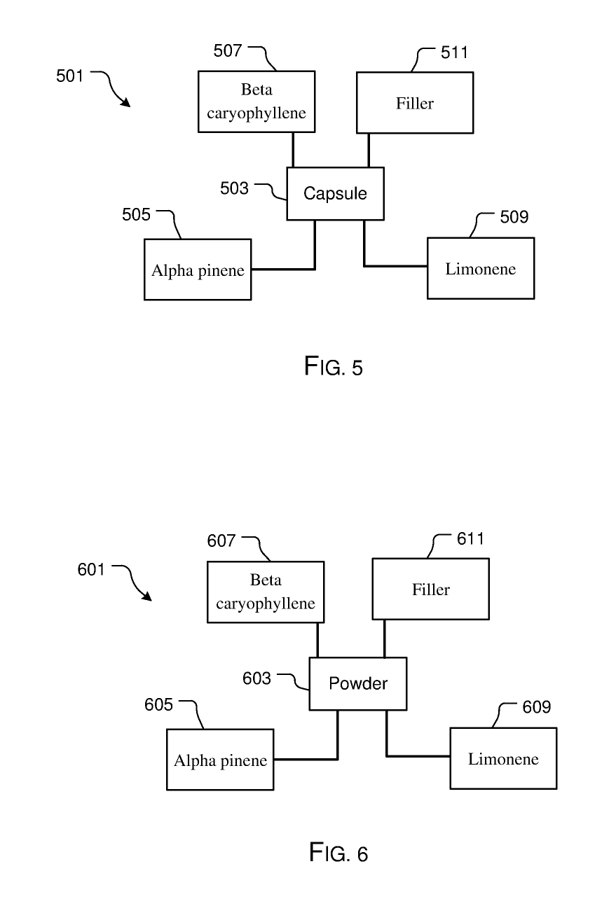

In FIG. 5, another simplified schematic 501 shows another embodiment of the product associated with method 201, wherein the product is a capsule 503, and wherein the terpenes 505, 507, 509 are encapsulated along with a filler 511. It should be appreciated that this configuration allows for a user to easily take a capsule before or after ingesting a cannabis product, as desired by the user. It should be appreciated that filler 511 can be an inactive ingredient for the purpose of long term stabilization and/or bulking of the product. Such fillers can include binders such as saccharides, gelatin, or polymers. Additional ingredients, such as antiadherents, coatings, and disintegrants can be added as necessary for manufacturing purposes.

In FIG. 6, a simplified schematic 601 shows another embodiment of the product associated with method 201, wherein the product is a powder 603 and wherein the terpenes 605, 607, 609 are added to powder 603 along with a filler 611. It should be understood that powder 603 can be a cannabis infused, or alternatively can be merely an ingestible powder to be added to a drink or other product.

It should be appreciated that one of the unique features believed characteristic of the present application is the method of creating a product for ingestion, wherein the product contains alpha pinene, beta caryophyllene, and limonene. It should be appreciated that the resulting product is suitable for use before, during, or after the ingestion of cannabis to help the user combat cannabis induced paranoia and/or anxiety. In the preferred embodiment, the combination of terpenes will comprise approximately 50% of the overall composition of the product, however, it should be appreciated that adjustments can be made. One alternative embodiment comprises approximately 75% of the overall composition of the product, with approximately 25% being a filler.

The particular embodiments disclosed above are illustrative only, as the embodiments may be modified and practiced in different but equivalent manners apparent to those skilled in the art having the benefit of the teachings herein. It is therefore evident that the particular embodiments disclosed above may be altered or modified, and all such variations are considered within the scope and spirit of the application. Accordingly, the protection sought herein is as set forth in the description. Although the present embodiments are shown above, they are not limited to just these embodiments, but are amenable to various changes and modifications without departing from the spirit thereof.

What is claimed is:

1. A system for combatting cannabis induced paranoia and anxiety, the system comprising:
a product to be ingested by a user; and
a terpene mixture to be incorporated into the product, the terpene mixture having:
an amount of alpha pinene;
an amount beta caryophyllene; and
an amount of limonene;
wherein the terpene mixture is integrally mixed into the product at a predetermined percentage; and
wherein the product is configured to be ingested to combat cannabis induced paranoia and/or anxiety.

2. The system of claim 1, wherein the predetermined percentage is approximately 50%.

3. The system of claim 1, wherein the predetermined percentage is approximately 75%.

4. The system of claim 1, wherein the amount of alpha pinene, the amount of beta caryophyllene, and the amount of limonene are approximately equal.

5. The system of claim 1, wherein the terpene mixture comprises natural extracts.

6. The system of claim 1, wherein the terpene mixture is organically derived.

7. The system of claim 1, wherein the terpene mixture is synthetically derived.

8. The system of claim 1, wherein the product is an edible cannabis product.

9. The system of claim 1, wherein the product further comprises:
a capsule configured to be ingested and filled with approximately 50% of the terpene mixture and filled a remaining 50% with a filler.

10. The system of claim 1, wherein the product is a cannabis distillate.

11. The system of claim 1, wherein the product further comprises:
a powder configured to be ingested and filled having approximately 50% of the terpene mixture in a powered form and a remaining 50% with a filler.

12. A method of creating a product to combat cannabis induced paranoia and anxiety, the method comprising:
selecting a predetermined amount of three terpenes, the three terpenes being alpha pinene, beta caryophyllene, and limonene; and
mixing the predetermined amount of three terpenes into an ingestible product.

13. The method of claim 12, further comprising:
premixing the three terpenes to create a terpene mixture.

14. The method of claim 12, wherein the predetermined amount of three terpenes comprises an equal ratio of the three terpenes.

15. The method of claim 12, wherein the predetermined amount of three terpenes comprises approximately 50% of the ingestible product.

16. The method of claim 12, wherein the predetermined amount of three terpenes comprises approximately 75% of the ingestible product.

17. The method of claim 12, further comprising:
adding a filler to the ingestible product;
wherein the filler is an edible filler.

18. The method of claim 12, further comprising:
encapsulating the ingestible product and the three terpenes into a capsule.

19. The method of claim 12, wherein the ingestible product is a powder.

20. The method of claim 12, wherein the ingestible product is an edible cannabis product.
